(19) United States
(12) Patent Application Publication
(10) Pub. No.: US 2010/0241091 A1
(43) Pub. Date: Sep. 23, 2010

(54) Sputum collecting device

(75) Inventor: Tan Wu, Yuenlin Hsien (TW)

Correspondence Address: CHARLES E. BAXLEY, ESQUIRE, 90 JOHN STREET, SUITE 309, NEW YORK, NY, 10038, US

(73) Assignees: Mr. Tan Wu (Owners in common 1/2); Yung Kang Medical Device & Technologies Co., Ltd. (Owners in common 1/2)

(21) Appl. No.: 12/383,146

(22) Filed: Mar. 20, 2009

(51) Int. Cl. A61M 1/00

(52) U.S. Cl. 604/319

(57) ABSTRACT

A sputum collecting device includes a bag attached to a cover and disposed in a container for receiving and collecting a sputum and for forming a peripheral space between the container and the bag, the cover includes an inlet for guiding the sputum from a patient into the bag and includes a tubular member communicative with the container and the peripheral space that is formed between the container and the bag, and a vacuuming source for drawing the air in the bag and the peripheral space that is formed between the container and the bag, and for drawing the sputum through the inlet of the cover and into the bag, and then for collecting the sputum in the bag.

### BACKGROUND OF THE INVENTION

1. Field of the Invention

The present invention relates to a sputum collecting device, and more particularly to a sputum collecting device for medical use and including a structure for allowing the sputum, the phlegm, or the other fluid or liquid to be easily induced and collected in an inner bag that can be easily discarded.

2. Description of the Prior Art

Typical sputum collecting devices comprise a container, and a funnel attached to the container and having an enlarged mouth for receiving or collecting the sputum, the phlegm, or the other fluid or liquid from the patient or the like.

For example, U.S. Pat. No. 4,589,548 to Fay discloses one of the typical sputum collecting apparatuses comprising a sputum container having an open end and a closed end, and a funnel having a mouth at one end for receiving the sputum and a discharge portion at the other end for discharging the sputum.

However, when the sputum, the phlegm, or the other fluid or liquid is received or collected in the sputum container, the germs, pathogenic bacteria or the like contained in the sputum container may be exposed through the open end of the sputum container and may pollute the environment, particularly the hospital.

U.S. Pat. No. 4,771,486 to Gutierrez et al. discloses another typical sputum specimen collecting device comprising a circular cup having a wall portion which tapers inwardly from top to bottom, and a separation plate having an elliptical planar configuration disposed in the cup for retracting and holding sputum.

However, similarly, the germs, pathogenic bacteria or the like contained in the sputum cup may be exposed through the open upper end of the sputum cup and may pollute the environment, particularly the hospital.

U.S. Pat. No. 6,984,214 to Fowler-Hawkins discloses a further typical sputum inducing and collecting device comprising a housing, a reed disposed in the housing, and an acoustical resistance for coupling a patient lung cavity to the audio shockwave, thereby vibrating the patient's lung cavity to thin lung secretions.

However, the typical sputum inducing and collecting device may not be used to easily induce or to receive or to collect the sputum in the housing, and the sputum may not be easily removed or disengaged form the housing.

The present invention has arisen to mitigate and/or obviate the afore-described disadvantages of the conventional sputum collecting devices.

### SUMMARY OF THE INVENTION

The primary objective of the present invention is to provide a sputum collecting device including a structure for allowing the sputum, the phlegm, or the other fluid or liquid to be easily induced and collected in an inner bag that can be easily discarded.

In accordance with one aspect of the invention, there is provided a sputum collecting device comprising a container including a chamber formed therein, and including an upper portion and a lower portion, a cover attached onto the upper portion of the container, a bag attached to the cover and disposed in the chamber of the container for receiving and collecting a sputum and for forming a peripheral space between the container and the bag, the cover including an inlet communicative with the bag for guiding the sputum from a patient into the bag, and including a tubular member communicative with the chamber of the container and the peripheral space that is formed between the container and the bag, and a vacuuming source for drawing the air in the bag and the peripheral space that is formed between the container and the bag, and for drawing the sputum through the inlet of the cover and into the bag, and then for collecting the sputum in the bag, and for allowing the bag and the sputum to be easily and quickly discarded.

The cover includes a check valve attached to the inlet of the cover for limiting the sputum to flow into the bag only and for preventing the sputum from flowing out through the inlet of the cover.

The cover includes a filter device attached to the tubular member and disposed in the chamber of the container and disposed in the bag for filtering the sputum and for preventing the sputum from flowing out through the tubular member of the cover and for preventing the germs contained in the sputum from being exposed and from polluting the environment, particularly the hospital. The cover includes a mouth formed therein. The cover includes a lid detachably attached to the mouth of the cover.

The container includes an outlet device communicative with the peripheral space that is formed between the container and the bag and coupled to the vacuuming source. The outlet device includes an outlet port coupled to the vacuuming source. The outlet device includes a fastener engaged through the container for securing the outlet device to the container.

The outlet device includes a conduit formed therein, and the fastener includes a bore formed therein for communicating the conduit of the outlet device with the peripheral space that is formed between the container and the bag. The fastener includes a head disposed and located in the peripheral space and engaged between the container and the bag for spacing the bag from the container and for allowing the air to be suitably drawn or vacuumed out through the outlet port of the outlet device.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is an exploded view of a sputum collecting device in accordance with the present invention;

FIG. 2 is a perspective view of the sputum collecting device;

FIG. 3 is a top plan schematic view of the sputum collecting device;

FIG. 4 is a cross sectional view of the sputum collecting device taken along lines 4-4 of FIG. 3;

FIG. 5 is a side plan schematic view illustrating the operation of the sputum collecting device;

FIG. 6 is another exploded view similar to FIG. 1, illustrating the other arrangement of the sputum collecting device;

FIG. 7 is a perspective view of the sputum collecting device as shown in FIG. 6;

FIG. 8 is a side plan schematic view of the sputum collecting device as shown in FIGS. 6 and 7;

FIG. 9 is a top plan schematic view of the sputum collecting device as shown in FIGS. 6-8;

FIG. 10 is a rear plan schematic view of the sputum collecting device as shown in FIGS. 6-9;

FIG. 11 is a cross sectional view of the sputum collecting device taken along lines 11-11 of FIG. 10;

FIG. 12 is a further exploded view similar to FIGS. 1 and 6, illustrating the further arrangement of the sputum collecting device;

FIG. 13 is a still further exploded view of the sputum collecting device as shown in FIG. 12;

FIG. 14 is a perspective view of the sputum collecting device as shown in FIGS. 12-13, in which one half of the sputum collecting device has been cut off for showing the inner structure of the sputum collecting device;

FIG. 15 is a top plan schematic view of the sputum collecting device as shown in FIGS. 12-14; and

FIG. 16 is a cross sectional view of the sputum collecting device taken along lines 16-16 of FIG. 15.

### DETAILED DESCRIPTION OF THE PREFERRED EMBODIMENT

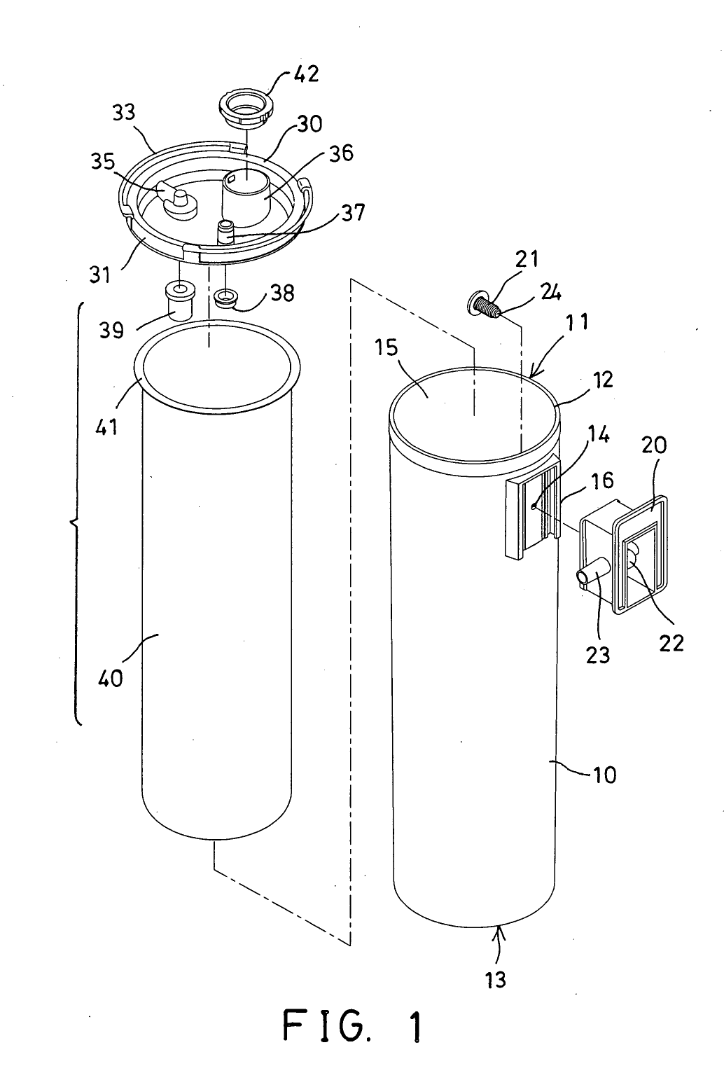

Referring to the drawings, and initially to FIGS. 1-4, a sputum collecting device in accordance with the present invention comprises an outer housing or container 10 having an open upper portion 11 formed or defined by a peripheral flange 12, and having a closed bottom end or lower portion 13, and having an aperture 14 formed through the upper portion 11 of the container 10 and communicating with the inner chamber 15 of the container 10, and having a retaining device 16 attached to the upper portion 11 of the container 10 and disposed or arranged around the aperture 14 of the container 10.

A cover 30 includes a peripheral lip 31 folded downwardly for forming or defining a downwardly opened peripheral channel 32 and for receiving or engaging with the upper peripheral flange 12 of the container 10 and for openably attaching or mounting the cover 30 onto the upper portion 11 of the container 10, the cover 30 includes one or more (such as two) hand grips 33 provided or formed on the outer or the upper portion of the cover 30 for selectively engaging the cover 30 onto the container 10 or for selectively opening or disengaging the cover 30 from the container 10.

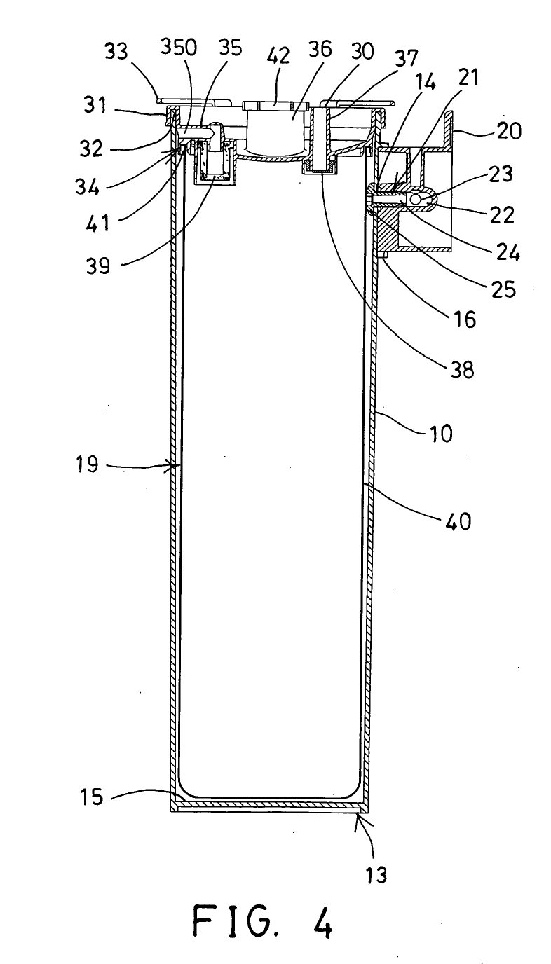

An envelope or flexible inner bag 40 is disposed within the inner chamber 15 of the container 10 for receiving or collecting the sputum, the phlegm, or the other fluid or liquid from the patient or the like, and includes an upper portion 41 attached or secured to the bottom portion 34 of the cover 30 for allowing the inner bag 40 to be sustained in the inner chamber 15 of the container 10 and for forming or defining a peripheral gap or space 19 between the container 10 and the inner bag 40 (FIG. 4), the cover 30 includes a tubular member 35 having a passage 350 formed therein and communicative with the inner chamber 15 of the container 10 and the outer peripheral portion of the cover 30, and thus for selectively communicating the inner chamber 15 of the container 10 with the peripheral space 19 that is formed between the container 10 and the inner bag 40.

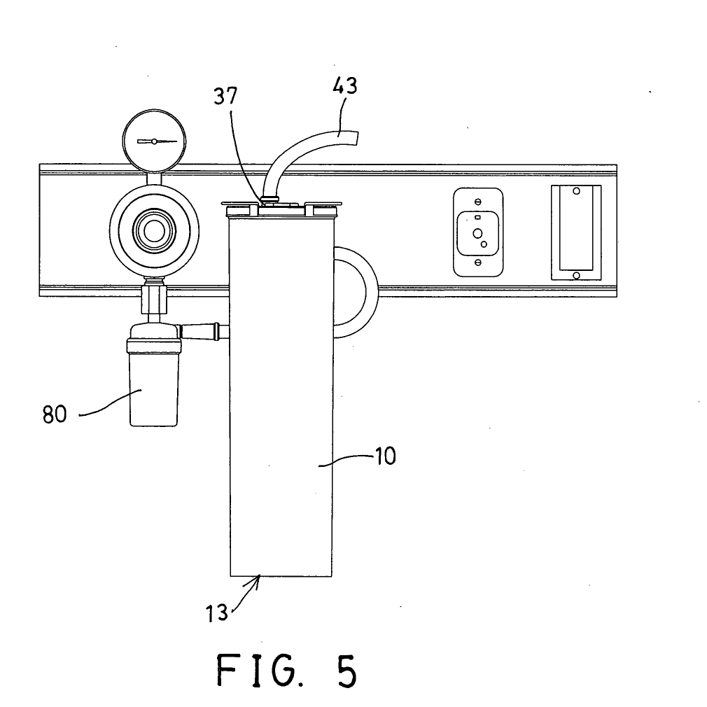

The cover 30 includes a mouth 36 formed therein and enclosable with a lid 42 which may be removed or disengaged form the mouth 36 of the cover 30 for allowing the sputum, the phlegm, or the other fluid or liquid to be selectively removed or poured out through the mouth 36 of the cover 30, and the cover 30 includes an inlet 37 communicative with the inner portion of the inner bag 402, and coupled to a hose 43 (FIG. 5) which may be used for coupling to a patient and for inducing or vacuuming or drawing and collecting the sputum, the phlegm, or the other fluid or liquid from the patient or the like. A check valve 38 is attached to the bottom or inner portion of the inlet 37 for controlling or limiting the sputum to flow into the bag 40 only and for preventing the sputum from flowing out through the inlet 37 of the cover 30.

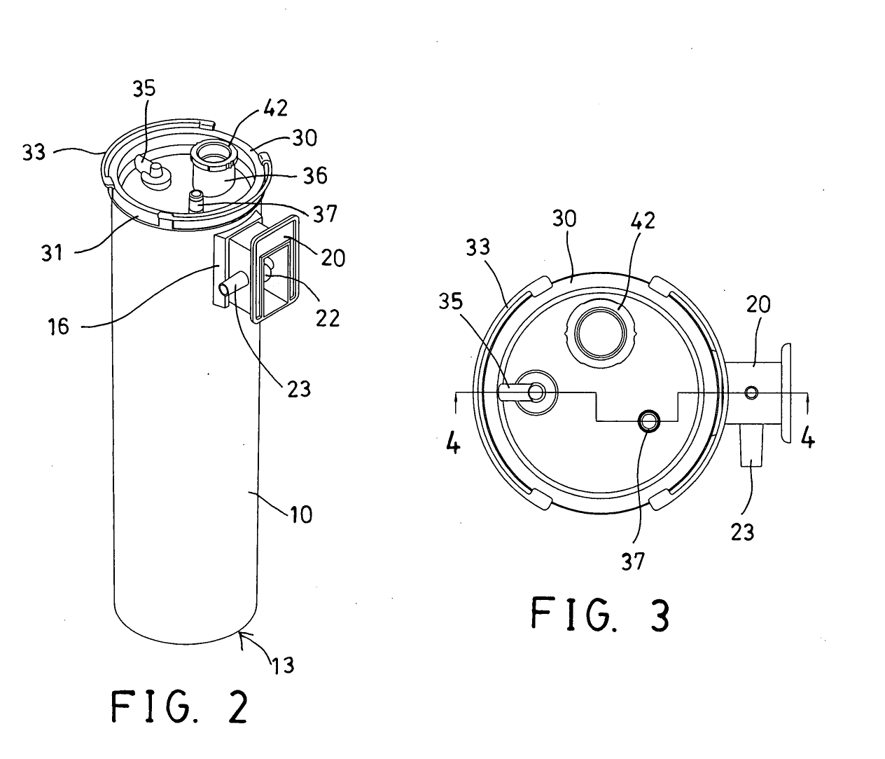

A filter device 39 is attached to the bottom or inner portion of the tubular member 35 and disposed or arranged within the inner chamber 15 of the container 10 and also within the bag 40 and for filtering the sputum and for preventing the sputum from flowing out through the tubular member 35 of the cover 30 and for preventing the sputum and the germs, the pathogenic bacteria or the like contained in the sputum from being exposed and from polluting the environment, particularly the hospital. An outlet device 20 is attached or secured to the upper portion 11 of the container 10, such as attached or secured to the retaining device 16 with a fastener 21 (FIGS. 2, 4) which is engaged through the aperture 14 of the container 10.

The outlet device 20 includes a conduit 22 formed or provided therein, and includes an outlet port 23 for coupling to an air system or pump or vacuuming source 80 (FIG. 5) which may draw or vacuum the air out of the conduit 22 of the outlet device 20. As shown in FIG. 5, the fastener 21 includes a bore 24 formed therein for communicating the conduit 22 of the outlet device 20 with the peripheral space 19 that is formed between the container 10 and the inner bag 40 and for allowing the air in the peripheral space 19 that is formed between the container 10 and the inner bag 40 to be drawn or vacuumed out through the conduit 22 and the outlet port 23 of the outlet device 20 by the vacuuming source 80. The fastener 21 includes an enlarged head 25 disposed or arranged and located in the peripheral space 19 and engaged between the container 10 and the inner bag 40 for suitably spacing the inner bag 40 from the container 10 and for allowing the air to be suitably drawn or vacuumed out through the outlet port 23 of the outlet device 20.

In operation, as shown in FIGS. 3-5. when the vacuuming source 80 is energized or operated, the sputum, the phlegm, or the other fluid or liquid from the patient may be suitably drawn or vacuumed through the hose 43 and the inlet 37 of the cover 30 and into the bag 40, and then collected in the bag 40. The air may be drawn out through the filter device 39, the passage 350 of the tubular member 35, the peripheral space 19 that is formed between the container 10 and the inner bag 40, the bore 24 of the fastener 21, the conduit 22 of the outlet device 20, and then drawn out through the outlet port 23 of the outlet device 20 by the vacuuming source 80 for allowing the sputum, the phlegm, or the other fluid or liquid to be suitably drawn and collected in the bag 40, and for preventing the sputum and the germs, the pathogenic bacteria or the like contained in the sputum from being exposed and from polluting the environment, particularly the hospital, the bag 40 and the sputum may then be easily and quickly removed or disengaged form the container 10 when required.

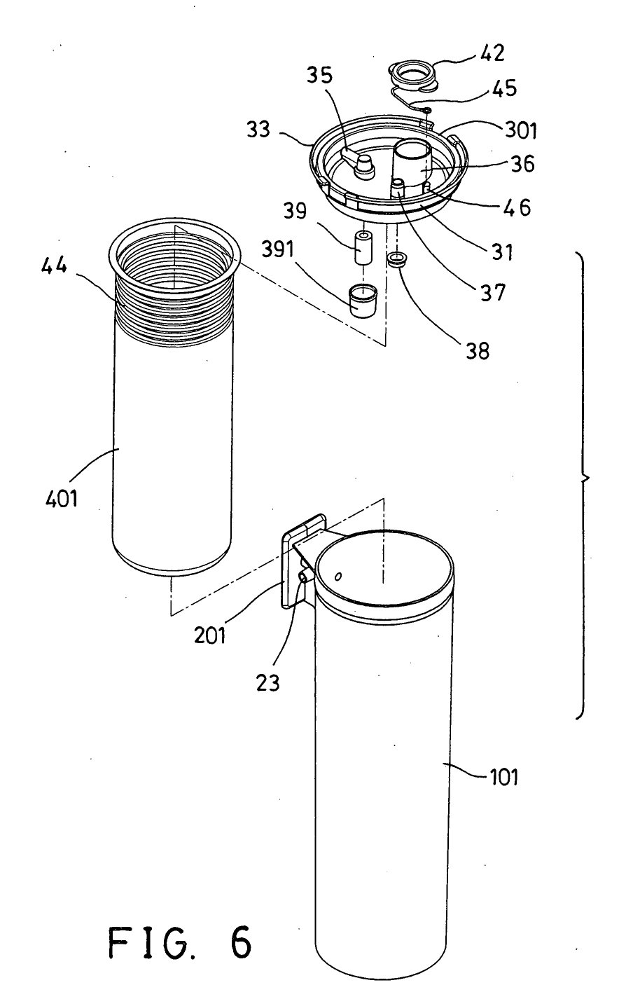

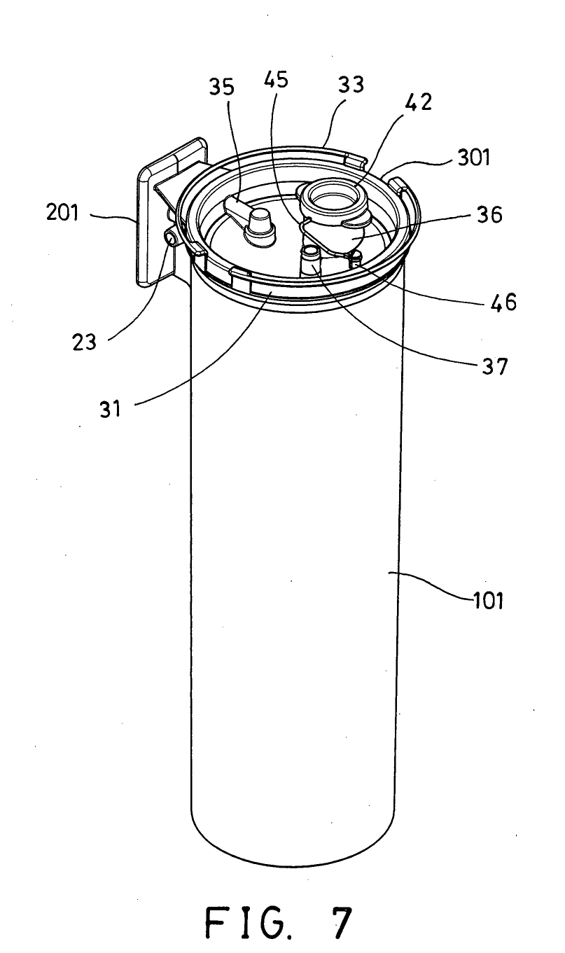

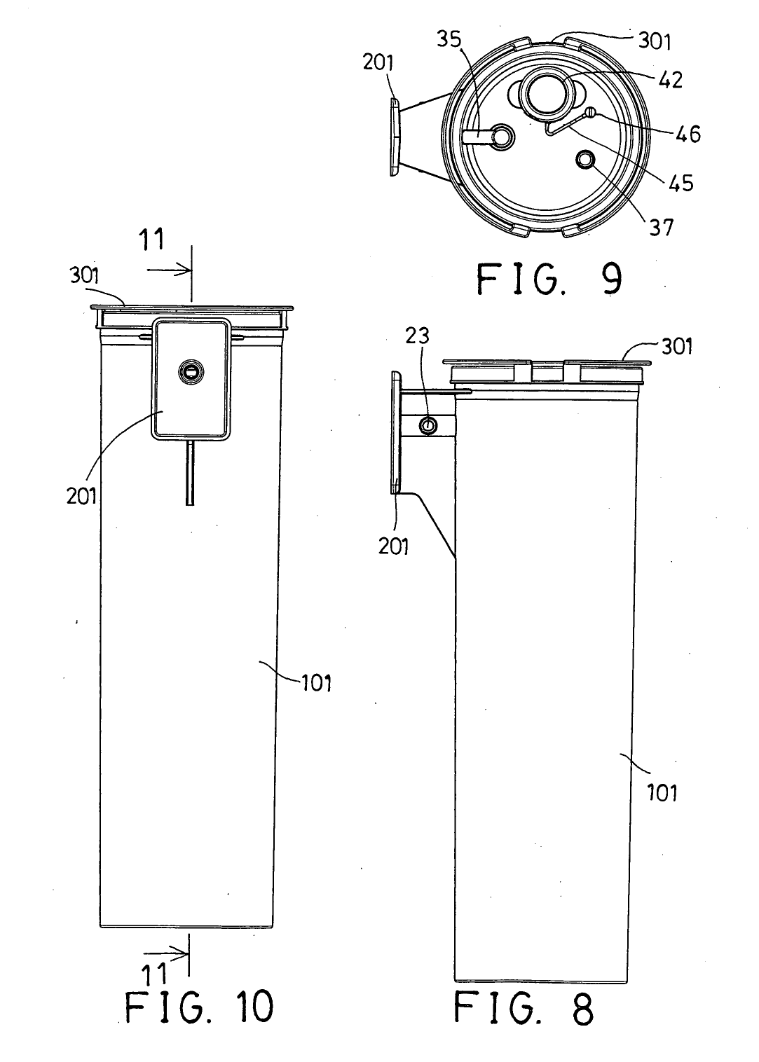

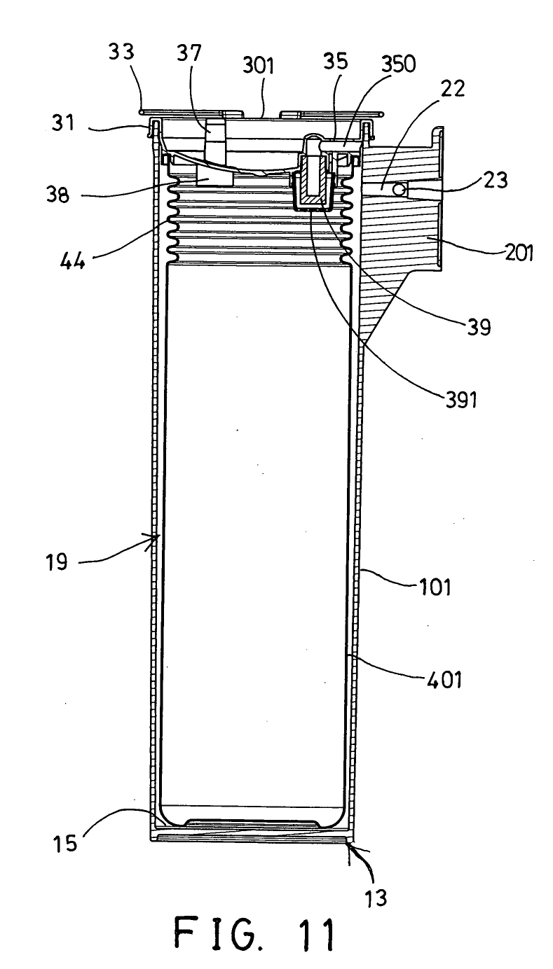

Alternatively, as shown in FIGS. 6-11, the outlet device 201 may be formed integral with the container 101 and the conduit 22 of the outlet device 201 is directly communicative with the peripheral space 19 that is formed between the container 101 and the inner bag 401 for allowing the air in the peripheral space 19 that is formed between the container 101 and the inner bag 401 to be drawn or vacuumed out through the conduit 22 of the outlet device 201 by the vacuuming source. The bag 401 may further include a serrated or bellows portion 44 for allowing the air to be suitably drawn out through the conduit 22 and the outlet port 23 of the outlet device 201. The lid 42 may further include a link 45 detachably coupled to an anchor 46 for coupling the lid 42 to the cover 301 and for preventing the lid 42 from being disengaged form the cover 301 inadvertently. A protective shield 391 may further be provided and attached to the bottom or inner portion of the cover 301 for shielding or protecting the filter device 39.

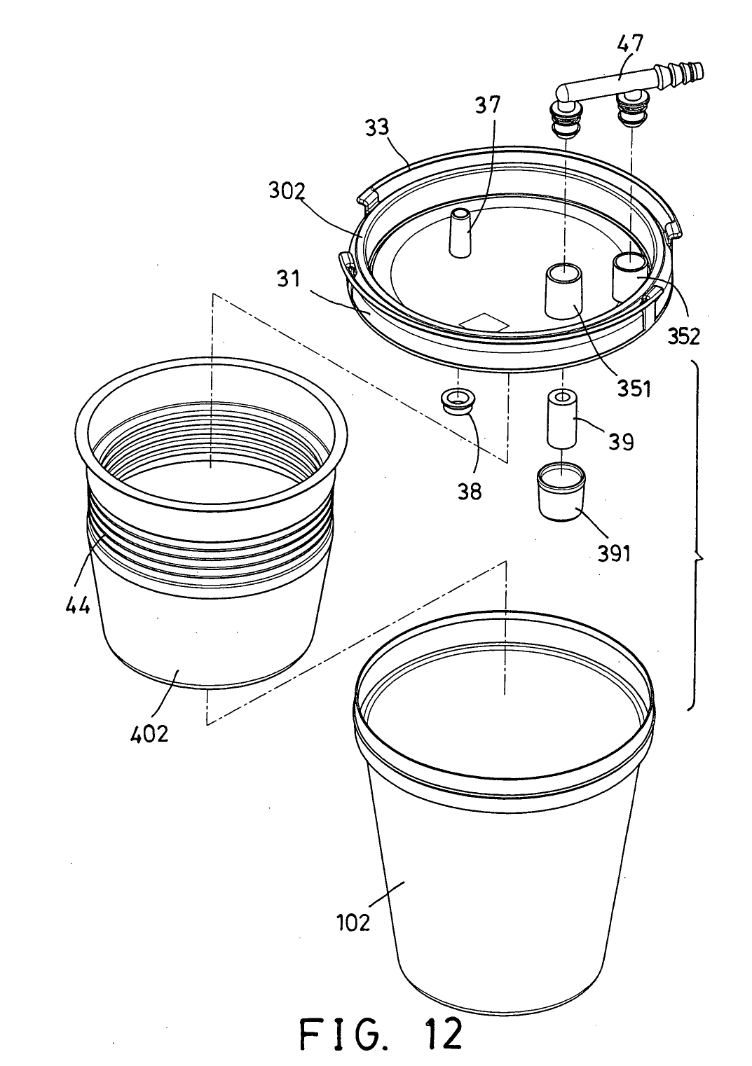

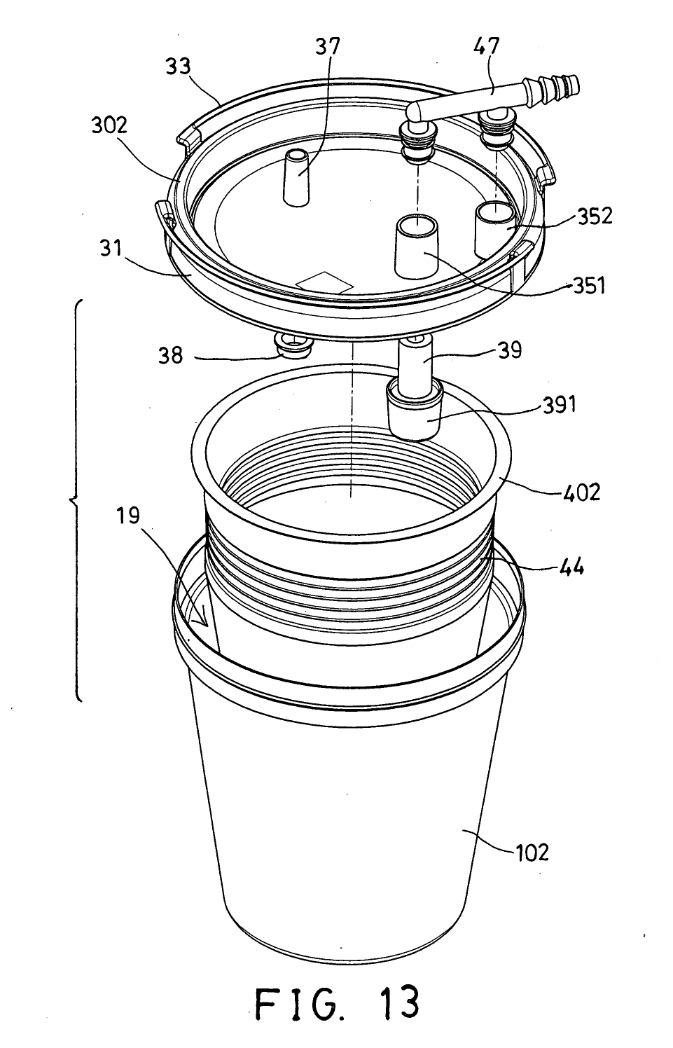

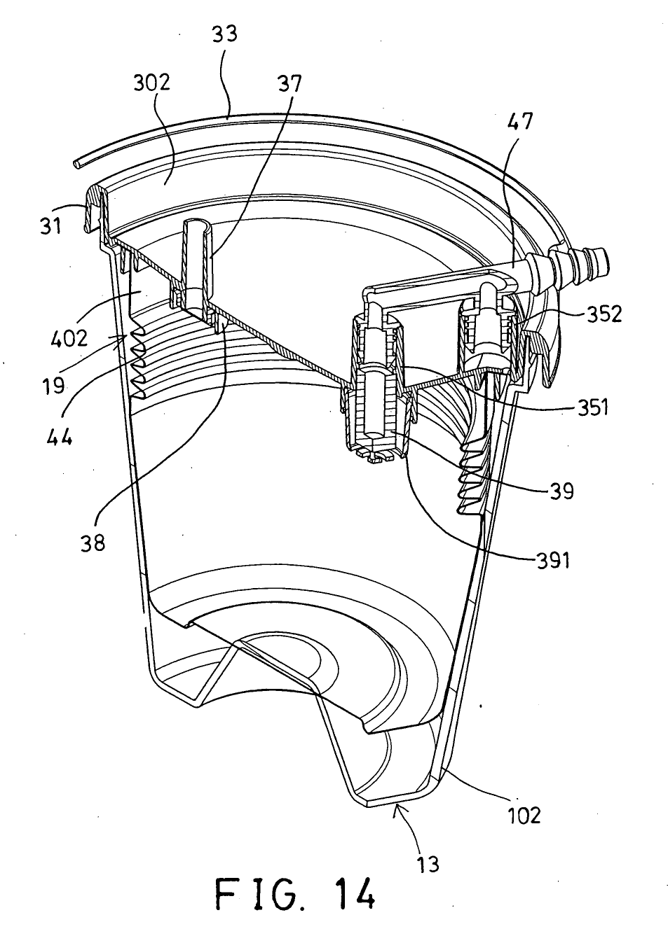

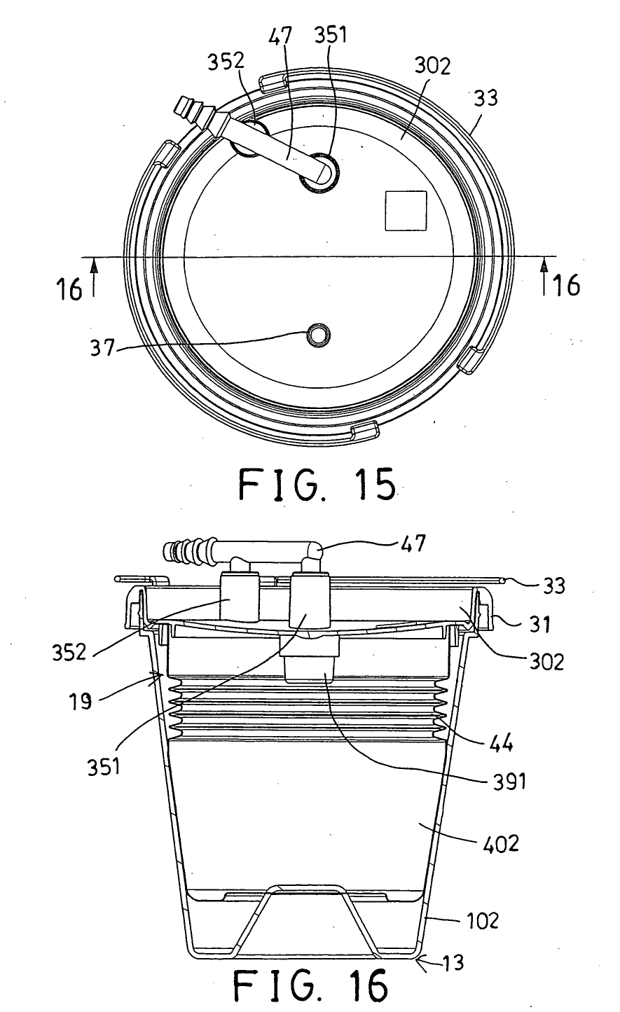

Further alternatively, as shown in FIGS. 12-16, the bag 402 may also be disposed within the container 102 and may also include a serrated or bellows portion 44, and the cover 302 may include two tubular members 351, 352 communicating with the inner portion of the inner bag 402 and the peripheral space 19 that is formed between the container 102 and the inner bag 402 respectively, the filter device 39 and the protective shield 391 may also be provided and attached to the bottom or inner portion of the cover 302 and coupled to the tubular member 351, and a coupler 47 may be coupled to the tubular members 351, 352 for coupling to the air system or pump or vacuuming source 80 (FIG. 5) which may directly draw or vacuum the air out of the inner portion of the inner bag 402 and the peripheral space 19 that is formed between the container 102 and the inner bag 402.

Accordingly, the sputum collecting device in accordance with the present invention includes a structure for allowing the sputum, the phlegm, or the other fluid or liquid to be easily induced and collected in the inner bag that can be easily discarded.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A sputum collecting device comprising:
a container including a chamber formed therein, and including an upper portion and a lower portion,
a cover attached onto said upper portion of said container,
a bag attached to said cover and disposed in said chamber of said container for receiving and collecting a sputum and for forming a peripheral space between said container and said bag,
said cover including an inlet communicative with said bag for guiding the sputum from a patient into said bag, and including a tubular member communicative with said chamber of said container and said peripheral space that is formed between said container and said bag, and
a vacuuming source for drawing the air in said bag and said peripheral space that is formed between said container and said bag, and for drawing the sputum through said inlet of said cover and into said bag.

2. The sputum collecting device as claimed in claim 1, wherein said cover includes a check valve attached to said inlet of said cover for limiting the sputum to flow into said bag only and for preventing the sputum from flowing out through said inlet of said cover.

3. The sputum collecting device as claimed in claim 1, wherein said cover includes a filter device attached to said tubular member and disposed in said chamber of said container and disposed in said bag.

4. The sputum collecting device as claimed in claim 1, wherein said cover includes a mouth formed therein.

5. The sputum collecting device as claimed in claim 4, wherein said cover includes a lid detachably attached to said mouth of said cover.

6. The sputum collecting device as claimed in claim 1, wherein said container includes an outlet device communicative with said peripheral space that is formed between said container and said bag and coupled to said vacuuming source.

7. The sputum collecting device as claimed in claim 6, wherein said outlet device includes an outlet port coupled to said vacuuming source.

8. The sputum collecting device as claimed in claim 6, wherein said outlet device includes a fastener engaged through said container for securing said outlet device to said container.

9. The sputum collecting device as claimed in claim 8, wherein said outlet device includes a conduit formed therein, and said fastener includes a bore formed therein for communicating said conduit of said outlet device with said peripheral space that is formed between said container and said bag.

10. The sputum collecting device as claimed in claim 9, wherein said fastener includes a head disposed and located in said peripheral space and engaged between said container and said bag for spacing said bag from said container.
